(19) United States
(12) Patent Application Publication
(10) Pub. No.: US 2023/0331846 A1
(43) Pub. Date: Oct. 19, 2023

(54) Canine PD-1-Binding Polypeptides and Uses Thereof

(71) Applicant: Inhibrx, Inc., La Jolla, CA (US)

(72) Inventors: Rajay Pandit, San Diego, CA (US); John C. Timmer, San Diego, CA (US); Brendan P. Eckelman, Encinitas, CA (US); Quinn Deveraux, La Jolla, CA (US)

(73) Assignee: Inhibrx, Inc., La Jolla, CA (US)

(21) Appl. No.: 17/922,569

(22) PCT Filed: May 3, 2021
(86) PCT No.: PCT/US2021/030476

§ 371 Date: Oct. 31, 2022

Related U.S. Application Data
(60) Provisional application No. 63/019,817, filed on May 4, 2020.

(51) Int. Cl. C07K 16/28; C12N 5/0783

(52) U.S. Cl. CPC C07K 16/2818; C12N 5/0636; A61K 39/00; C07K 2317/20; C07K 2317/52; C07K 2317/569

(57) ABSTRACT

Provided herein are VHH-containing polypeptides that bind canine PD-1. In some embodiments, VHH-containing polypeptides that bind and antagonize canine PD-1 are provided. Uses of the VHH-containing polypeptides are also provided.

### FIELD

The present invention relates to canine PD-1-binding polypeptides, and methods of using canine PD-1-binding polypeptides to modulate the biological activity of canine PD-1. Such methods include, but are not limited to, methods of treating cancer. In some embodiments, the canine PD-1-binding polypeptides are multivalent canine PD-1-binding polypeptides.

### BACKGROUND

Tumor infiltrating lymphocytes often contain tumor reactive T-cells and NK cells that are suppressed by immune checkpoints. Programmed cell death protein 1 (PD-1), also known as CD279, is expressed on activated T-cells. PD-1 inhibits T-Cell Receptor signaling, T-cell proliferation, and natural killer (NK) cell antitumor activity when engaged with PD-L1 (CD274) or PD-L2 (CD273) on adjacent cells in the tumor microenvironment. Antibodies that bind PD-1 and decrease and/or block binding of PD-L1 or PD-L2 to PD-1 have shown clinical benefit in a variety of cancer types.

Therefore, there exists a therapeutic need for more potent antibodies that bind canine PD-1.

### SUMMARY

Provided herein are polypeptides comprising at least one VHH domain that binds canine PD-1. In some embodiments, the polypeptide comprises a canine Fc region. In some embodiments, at least one VHH domain comprises a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8. In some embodiments, at least one VHH domain comprises a CDR1 comprising the amino acid sequence of SEQ ID NO: 22, a CDR2 comprising the amino acid sequence of SEQ ID NO: 23, and a CDR3 comprising the amino acid sequence of SEQ ID NO: 24. In some embodiments, at least one VHH domain comprises a CDR1 comprising the amino acid sequence of SEQ ID NO: 25, a CDR2 comprising the amino acid sequence of SEQ ID NO: 26, and a CDR3 comprising the amino acid sequence of SEQ ID NO: 27. In some embodiments, at least one VHH domain comprises a CDR1 comprising the amino acid sequence of SEQ ID NO: 28, a CDR2 comprising the amino acid sequence of SEQ ID NO: 29, and a CDR3 comprising the amino acid sequence of SEQ ID NO: 30. In some embodiments, at least one VHH domain comprises the amino acid sequence of SEQ ID NO: 9. In some embodiments, at least one VHH domain comprises the amino acid sequence of SEQ ID NO: 10. In some embodiments, at least one VHH domain comprises the amino acid sequence of SEQ ID NO: 11. In some embodiments, at least one VHH domain comprises the amino acid sequence of SEQ ID NO: 12. In some embodiments, at least one VHH domain comprises the amino acid sequence of SEQ ID NO: 13. In some embodiments, at least one VHH domain comprises the amino acid sequence of SEQ ID NO: 2, 3, 4, or 5. In some embodiments, at least one VHH domain comprises a caninized version of the amino acid sequence of SEQ ID NO: 2, 3, 4, or 5. In some embodiments, each VHH domain is caninized.

In some embodiments, the polypeptide comprises at least one VHH domain comprising a CDR1 comprising the amino acid sequence of SEQ ID NO: 6, a CDR2 comprising the amino acid sequence of SEQ ID NO: 7, a CDR3 comprising the amino acid sequence of SEQ ID NO: 8, and an amino acid sequence having at least 90%, 95%, 96%, 97%, 98%, 99%, or 100% identity an amino acid sequence selected from SEQ ID NOs: 9-13.

In some embodiments, the polypeptide comprises two VHH domains. In some embodiments, the polypeptide comprises three VHH domains. In some embodiments, each VHH domain binds canine PD-1. In some embodiments, each VHH domain comprises a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8; or a CDR1 comprising the amino acid sequence of SEQ ID NO: 22, a CDR2 comprising the amino acid sequence of SEQ ID NO: 23, and a CDR3 comprising the amino acid sequence of SEQ ID NO: 24; or a CDR1 comprising the amino acid sequence of SEQ ID NO: 25, a CDR2 comprising the amino acid sequence of SEQ ID NO: 26, and a CDR3 comprising the amino acid sequence of SEQ ID NO: 27; or a CDR1 comprising the amino acid sequence of SEQ ID NO: 28, a CDR2 comprising the amino acid sequence of SEQ ID NO: 29, and a CDR3 comprising the amino acid sequence of SEQ ID NO: 30. In some embodiments, each VHH domain comprises a CDR1 comprising the amino acid sequence of SEQ ID NO: 6, a CDR2 comprising the amino acid sequence of SEQ ID NO: 7, a CDR3 comprising the amino acid sequence of SEQ ID NO: 8, and an amino acid sequence having at least 90%, 95%, 96%, 97%, 98%, 99%, or 100% identity an amino acid sequence selected from SEQ ID NOs: 9-13. In some embodiments, each VHH domain comprises the amino acid sequence of SEQ ID NO: 9, 10, 11, 12, or 13. In some embodiments, each VHH domain comprises the amino acid sequence of SEQ ID NO: 9, 10, 11, 12, or 13, or the amino acid sequence of SEQ ID NO: 2, 3, 4, or 5, or a caninized version of the amino acid sequence of SEQ ID NO: 2, 3, 4, or 5. In some embodiments, each VHH domain is caninized.

In some embodiments, the polypeptide comprises an Fc region. In some embodiments, the Fc region is a canine IgGB Fc region. In some embodiments, the Fc region comprises the amino acid sequence of SEQ ID NO: 19 or 20. In some such embodiments, provided herein is a polypeptide that binds canine PD-1 comprising the amino acid sequence of SEQ ID NO: 14, 15, 16, 17, or 18. In some embodiments, provided herein is a polypeptide that binds canine PD-1 consisting of the amino acid sequence of SEQ ID NO: 14, 15, 16, 17, or 18.

In various embodiments, the polypeptide provided herein forms a dimer under physiological conditions. In some such embodiments, the polypeptide comprises an Fc region.

In some embodiments, the polypeptide decreases or blocks binding of PD-L1 to PD-1 in vitro and/or in vivo. In some embodiments, the polypeptide decreases binding of PD-L1 to PD-1 in vitro by at least 50%, at least 60%, at least 70%, at least 80%, or at least 90%. In some embodiments, the polypeptide blocks binding of PD-L1 to PD-1 in vitro. In some embodiments, the polypeptide blocks binding of PD-L1 to PD-1 in vitro with an IC₅₀ less than 100 nM, less than 75 nM, less than 50 nM, less than 40 nM, less than 30 nM, less than 20 nM, or less than 10 nM.

In some embodiments, the polypeptide has reduced binding to canine Fc receptor components CD16, CD32, and/or CD64. In some embodiments, binding of the polypeptide to canine CD16, CD32, and/or CD64 is decreased relative to binding by a polypeptide comprising a wild type IgGB Fc region in vitro and/or in vivo. In some embodiments, binding of the polypeptide to CD16 in vitro is reduced by at least 1.5-fold or at least 2-fold. In some embodiments, binding of the polypeptide to CD32 or CD64 in vitro is reduced by at least 10,000-fold. In some embodiments, the polypeptide exhibits reduced complement activation and/or inflammation in vivo relative to a polypeptide comprising a wild type IgGB Fc region.

In various embodiments, the polypeptide comprising at least one VHH domain that binds canine PD-1 provided herein is an antagonist of canine PD-1 biological activity. In some embodiments, the polypeptide binds canine PD-1 with an affinity (K_{D}) of less than 100 nM, less than 50 nM, less than 25 nM, or less than 10 nM.

In some embodiments, pharmaceutical compositions are provided, comprising a polypeptide comprising at least one VHH domain that binds canine PD-1 provided herein and a pharmaceutically acceptable carrier.

In some embodiments, an isolated nucleic acid is provided that encodes a polypeptide comprising at least one VHH domain that binds canine PD-1 provided herein. In some embodiments, a vector is provided that comprises the nucleic acid. In some embodiments, a host cell comprising the nucleic acid or vector is provided. In some embodiments, a host cell is provided that expresses a polypeptide comprising at least one VHH domain that binds canine PD-1 provided herein. In some embodiments, a method of producing the polypeptide comprising at least one VHH domain that binds canine PD-1 is provided, comprising incubating the host cell under conditions suitable for expression of the polypeptide. In some embodiments, the method further comprises isolating the polypeptide.

In some embodiments, methods of treating cancer are provided, comprising administering to a subject with cancer a pharmaceutically effective amount of a polypeptide comprising at least one VHH domain that binds canine PD-1 provided herein. In some embodiments, the cancer is lymphoma, hemangiosarcoma, mast cell carcinoma, melanoma, osteosarcoma, or mammary cancer. In some embodiments, the cancer is high grade lymphoma, histiocytic sarcoma, malignant histiocytosis, urothelial carcinoma, or oral squamous cell carcinoma. In some embodiments, the cancer is selected from renal cell carcinoma, non-small cell lung cancer, basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; adenocarcinoma of the lung; squamous carcinoma of the lung; myeloma; neuroblastoma; oral cavity cancer; ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma; non-Hodgkin’s lymphoma; B-cell lymphoma; low grade/follicular non-Hodgkin’s lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom’s macroglobulinemia; chronic lymphocytic leukemia (CLL); acute lymphoblastic leukemia (ALL); Hairy cell leukemia; and chronic myeloblastic leukemia.

In some embodiments, the method of treating cancer further comprises administering an additional therapeutic agent. In some embodiments, the additional therapeutic agent is an anti-cancer agent. In some embodiments, the anti-cancer agent is selected from a chemotherapeutic agent, an anti-cancer biologic, radiation therapy, CAR-T therapy, and an oncolytic virus. In some embodiments, the additional therapeutic agent is an anti-cancer biologic. In some embodiments, the anti-cancer biologic is an agent that inhibits PD-1 and/or PD-L1. In some embodiments, the anti-cancer biologic is an agent that inhibits VISTA, gpNMB, B7H3, B7H4, HHLA2, CD73, CTLA4, or TIGIT. In some embodiments, the anti-cancer biologic is an antibody. In some embodiments, the anti-cancer biologic is a cytokine. In some embodiments, the anti-cancer agent is CAR-T therapy. In some embodiments, the anti-cancer agent is an oncolytic virus. In some embodiments, a method of treating cancer provided herein further comprises tumor resection and/or radiation therapy.

### BRIEF DESCRIPTION OF THE FIGURES

FIGS. 1A-1B show binding of antibodies comprising a VHH domain to 293FS cells that express canine PD-1.

FIGS. 2A-2B show binding of canine PD-L1 to 293FS cells that express canine PD-1 in the presence of increasing concentrations of antibodies comprising a VHH domain that binds to canine PD-1.

FIGS. 3A-3C show binding of antibodies comprising a wild type or mutant canine IgGB Fc region to Fc receptor component CD16 (FIG. 3A), CD32 (FIG. 3B), or CD64 (FIG. 3C).

FIGS. 4A-4B show activation of CD4 (FIG. 4A) and CD8 (FIG. 4B) T cells in the presence of increasing concentrations of antibodies comprising a VHH domain that binds canine PD-1 and a mutant canine IgGB Fc region.

FIGS. 5A-5 show mean plasma concentrations of an anti-PD-1 sdAb after first and second intravenous infusions of the sdAb to dogs. The error bars show the standard deviation from the mean for the three dogs in each group.

### DETAILED DESCRIPTION

Embodiments provided herein relate to canine PD-1-binding polypeptides that modulate the activity of canine PD-1 and their use in various methods of treating cancer. Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety. In case of any contradiction or conflict between material incorporated by reference and the expressly described content provided herein, the expressly described content controls.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991).

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a particular cancer or PD-1-related disorder). In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted qualifications to be “responsive”.

“Canine” in reference to a subject includes, but is not limited to, domestic dogs.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“PD-1” as used herein refers to any native, mature PD-1 that results from processing of an PD-1 precursor in a cell. The term includes PD-1 from any vertebrate source, including mammals such as canines and felines, unless otherwise indicated. The term also includes naturally-occurring variants of PD-1, such as splice variants or allelic variants. A nonlimiting exemplary canine PD-1 amino acid sequence is shown, e.g., in GenBank Accession No. BAO74171.1. See SEQ ID NO. 1.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to an PD-1 epitope is a sdAb or VHH-containing polypeptide that binds this epitope with greater affinity, avidity, more readily, and/or with greater duration than it binds to other PD-1 epitopes or non-PD-1 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

As used herein, the term “modulate” with regard to the activity of PD-1 refers to a change in the activity of PD-1. In some embodiments, “modulate” refers to a decrease in PD-1 activity compared to PD-1 in the absence of the modulator.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antigen-binding molecule specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antigen-binding molecule specific to the epitope binds. It is noted that, in some embodiments, not every one of the residues within the linear epitope need be directly bound (or involved in a bond) by the antigen-binding molecule. In some embodiments, linear epitopes can be from immunizations with a peptide that effectively consisted of the sequence of the linear epitope, or from structural sections of a protein that are relatively isolated from the remainder of the protein (such that the antigen-binding molecule can interact, at least primarily), just with that sequence section.

The terms “antibody” and “antigen-binding molecule” are used interchangeably in the broadest sense and encompass various polypeptides that comprise antibody-like antigen-binding domains, including but not limited to conventional antibodies (typically comprising at least one heavy chain and at least one light chain), single-domain antibodies (sdAbs, comprising just one chain, which is typically similar to a heavy chain), VHH-containing polypeptides (polypeptides comprising at least one heavy chain only antibody variable domain, or VHH), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, while the hinge mediates dimerization) and Fc regions (comprising hinge, CH2, and CH3, where the hinge mediates dimerization).

The term antibody also includes, but is not limited to, chimeric antibodies, humanized antibodies, caninized antibodies, felinized antibodies, and antibodies of various species such as camelid (including llama), shark, mouse, human, cynomolgus monkey, etc.

The terms “single domain antibody” and “sdAb” are used interchangeably herein to refer to an antibody having a domain, such as a pair of variable domains of heavy chains (or VHH), without a light chain.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH polypeptide may form a dimer. Nonlimiting structures of VHH-containing polypeptides include VHHₗ-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, when a VHH-containing polypeptide comprises an Fc, it forms a dimer. Thus, the structure VHH₁-VHH₂-Fe, if it forms a dimer, is considered to be tetravalent (i.e., the dimer has four VHH domains). Similarly, the structure VHH₁-VHH₂-VHH₃-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains (CH1, CH2, and CH3). Certain heavy chain constant regions also comprise a hinge between the CH1 and CH2 domains, and/or a CH4 domain. Of course, non-function-altering deletions and alterations within the domains are encompassed within the scope of the term “heavy chain constant region,” unless designated otherwise. Certain canine isotypes can be further subdivided into subclasses. For example, canine IgG antibodies include, but are not limited to, IgGA, IgGB, IgGC, and IgGD antibodies.

An “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is a canine IgGA, IgGB, IgGC, or IgGD Fc. In some embodiments, an Fc region is a canine IgGB Fc.

An “acceptor canine framework” as used herein is a framework comprising the amino acid sequence of a heavy chain variable domain (VH) framework derived from a canine immunoglobulin framework, as discussed herein. An acceptor canine framework derived from a canine immunoglobulin framework or a canine consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the canine frameworks in a single antigen binding domain, such as a VHH.

“Affinity” refers to the strength of the sum total of noncovalent interactions between a single binding site of a molecule (for example, an antibody or VHH-containing polypeptide) and its binding partner (for example, an antigen). The affinity or the apparent affinity of a molecule X for its partner Y can generally be represented by the dissociation constant (K_{D}) or the K_{D}-apparent, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D}-_{apparent.} K_{D}-ₐₚₚₐᵣₑₙₜ, as used herein, is the concentration of an antigen-binding molecule or antigen at which it is 50% of the antigen-binding molecule or antigen is bound to the antigen or antigen-binding molecule, respectively.

In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation (such as T cell proliferation), and inducing or increasing expression of cytokines.

The term “PD-1 activity” or “biological activity” of PD-1, as used herein, includes any biological effect or at least one of the biologically relevant functions of the PD-1 protein. In some embodiments, PD-1 activity includes the ability of PD-1 to interact or bind to PD-1 ligand (PD-1L) or PD-2 ligand (PD-2L). Additional, nonlimiting exemplary PD-1 activities include decreasing T-cell receptor (TCR) signaling, decreasing proliferation of CD4⁺ and/or CD8⁺ T cells, decreasing CK2 expression and/or activity in T cells, and increasing expression of E3 ubiquitin ligases of the CBL family in T cells.

An “agonist” or “activating” antibody (such as a sdAb or VHH-containing polypeptide) is one that increases and/or activates a biological activity of the target antigen. In some embodiments, the agonist antibody binds to an antigen and increases its biologically activity by at least about 20%, 40%, 60%, 80%, 85% or more.

An “antagonist”, a “blocking” or “neutralizing” antibody is one that decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” VHH-containing polypeptide refers to a VHH-containing polypeptide with one or more alterations in one or more CDRs compared to a parent VHH-containing polypeptide that does not possess such alterations, such alterations resulting in an improvement in the affinity of the VHH-containing polypeptide for antigen.

A “caninized VHH” as used herein refers to a VHH in which one or more framework regions have been substantially replaced with canine framework regions. In some instances, certain framework region (FR) residues of the canine immunoglobulin are replaced by corresponding non-canine residues. Furthermore, the caninized VHH can comprise residues that are found neither in the original VHH nor in the canine framework sequences, but are included to further refine and optimize VHH or VHH-containing polypeptide performance. In some embodiments, a caninized VHH-containing polypeptide comprises a canine Fc region or canine heavy chain constant region. As will be appreciated, a caninized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence canine Fc regions include a native sequence canine IgGA Fc region; native sequence canine IgGB Fc region; native sequence canine IgGC Fc region; and native sequence canine IgGD Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” refers to a receptor that binds to the Fc region of an antibody. In some embodiments, an FcyR is a native canine FcR. In some embodiments, an FcR is one which binds an IgG antibody (a gamma receptor) and includes receptors of the FcyRI, FcyRII, and FcyRIII subclasses, including allelic variants and alternatively spliced forms of those receptors. FcyRII receptors include FcyRIIA (an “activating receptor”) and FcyRIIB (an “inhibiting receptor”), which have similar amino acid sequences that differ primarily in the cytoplasmic domains thereof. Activating receptor FcyRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGNTM (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| | |
|---|---|
| Original Residue | Exemplary Substitutions |
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6^{®} cells (Crucell), and 293, such as 293FS, and CHO cells, and additional derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “subject” is used herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, a “subject” is in need of treatment for a disease or disorder. In some embodiments, the subject to receive the treatment has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Exemplary cancers include, but are not limited to: lymphoma, hemangiosarcoma, mast cell carcinoma, melanoma, osteosarcoma, mammary cancer, renal cell carcinoma, and non-small cell lung cancer, high grade lymphoma, histiocytic sarcoma, malignant histiocytosis, urothelial carcinoma, oral squamous cell carcinoma; basal cell carcinoma; biliary tract cancer, bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin’s and non-Hodgkin’s lymphoma, as well as B-cell lymphoma (including low grade/follicular non-Hodgkin’s lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom’s Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs’ syndrome.

The term “non-tumor cell” as used herein refers to a normal cells or tissue. Exemplary non-tumor cells include, but are not limited to: T-cells, B-cells, natural killer (NK) cells, natural killer T (NKT) cells, dendritic cells, monocytes, macrophages, epithelial cells, fibroblasts, hepatocytes, interstitial kidney cells, fibroblast-like synoviocytes, osteoblasts, and cells located in the breast, skeletal muscle, pancreas, stomach, ovary, small intestines, placenta, uterus, testis, kidney, lung, heart, brain, liver, prostate, colon, lymphoid organs, bone, and bone-derived mesenchymal stem cells. The term “a cell or tissue located in the periphery” as used herein refers to non-tumor cells not located near tumor cells and/or within the tumor microenvironment.

The term “cells or tissue within the tumor microenvironment” as used herein refers to the cells, molecules, extracellular matrix and/or blood vessels that surround and/or feed a tumor cell. Exemplary cells or tissue within the tumor microenvironment include, but are not limited to: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells (Treg cells); macrophages; neutrophils; myeloid-derived suppressor cells (MDSCs) and other immune cells located proximal to a tumor. Methods for identifying tumor cells, and/or cells/tissues located within the tumor microenvironment are well known in the art, as described herein, below.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “an immune response” is meant to encompass cellular and/or humoral immune responses that are sufficient to inhibit or prevent onset or ameliorate the symptoms of disease (for example, cancer or cancer metastasis). “An immune response” can encompass aspects of both the innate and adaptive immune systems.

As used herein, “treatment” is an approach for obtaining beneficial or desired clinical results. “Treatment” as used herein, covers any administration or application of a therapeutic for disease in a mammal. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, “delaying development of a disease” means to defer, hinder, slow, retard, stabilize, suppress and/or postpone development of the disease (such as cancer). This delay can be of varying lengths of time, depending on the history of the disease and/or subject being treated. As is evident to one skilled in the art, a sufficient or significant delay can, in effect, encompass prevention, in that the subject does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the subject, and the ability of the substance/molecule, agonist or antagonist to elicit a desired response in the subject. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effect of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the subject.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary PD-1-Binding Polypeptides

Antagonist canine PD-1-binding polypeptides are provided herein. In various embodiments, the antagonist PD-1-binding polypeptides comprise at least one VHH domain that binds canine PD-1. In some embodiments, an antagonist PD-1-binding polypeptide provided herein comprises one, two, three, four, five, or six VHH domains that bind canine PD-1. In some embodiments, an antagonist canine PD-1-binding polypeptide provided herein comprises one, two, three, or four VHH domains that bind canine PD-1. Such canine PD-1-binding polypeptides may comprise one or more additional VHH domains that bind one or more target proteins other than canine PD-1.

In some embodiments, an antagonist canine PD-1-binding polypeptide comprises (i) at least one VHH domain that binds canine PD-1 and (ii) an Fc region. In some embodiments, an antagonist canine PD-1-binding polypeptide provided herein comprises (i) one, two, three, or four VHH domains that bind canine PD-1 and (ii) an Fc region. In some embodiments, an Fc region mediates dimerization of the canine PD-1-binding polypeptide at physiological conditions such that a dimer is formed that doubles the number of canine PD-1 binding sites. For example, a canine PD-1-binding polypeptide comprising an Fc region and three VHH domains that bind canine PD-1 is trivalent as a monomer, but at physiological conditions, the Fc region may mediate dimerization, such that the canine PD-1-binding polypeptide exists as a hexavalent dimer under such conditions.

In various embodiments, a VHH domain that binds canine PD-1 comprises a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8. In some embodiments, the VHH domain is caninized.

In some embodiments, a VHH domain that binds canine PD-1 may be caninized. Caninized antibodies (such as VHH-containing polypeptides) are useful as therapeutic molecules because caninized antibodies reduce or eliminate the canine immune response to non-canine antibodies, which can result in an immune response to an antibody therapeutic, and decreased effectiveness of the therapeutic. Generally, a caninized antibody comprises one or more variable domains in which CDRs (or portions thereof) are derived from a non-canine antibody, and FRs (or portions thereof) are derived from canine antibody sequences. A caninized antibody optionally will also comprise at least a portion of a canine constant region. In some embodiments, some FR residues in a caninized antibody are substituted with corresponding residues from a non-canine antibody (for example, the antibody from which the CDR residues are derived), for example, to restore or improve antibody specificity or affinity.

Canine framework regions that can be used for caninization include but are not limited to: framework regions selected using the “best-fit” method (see, for example, Sims et al. (1993) J. Immunol. 151 :2296); framework regions derived from the consensus sequence of canine antibodies of a particular subgroup of heavy chain variable regions (see, for example, Carter et al. (1992) Proc. Natl. Acad. Sci. USA, 89:4285; and Presta et al. (1993) J. Immunol, 151:2623); canine mature (somatically mutated) framework regions or canine germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271 :22611-22618). Typically, the FR regions of a VHH are replaced with canine FR regions to make a caninized VHH. In some embodiments, certain FR residues of the canine FR are replaced in order to improve one or more properties of the caninized VHH. VHH domains with such replaced residues are still referred to herein as “caninized.”

In some embodiments, a VHH domain that binds canine PD-1 comprises a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8.

In some embodiments, a canine PD-1-binding polypeptide comprises at least one VHH domain comprising the amino acid sequence of SEQ ID NO: 9, 10, 11, 12, or 13. In some embodiments, an PD-1-binding polypeptide comprises one, two, three, or four VHH domains comprising the independently selected amino acid sequence of SEQ ID NO: 9, 10, 11, 12, or 13.

In some embodiments, a canine PD-1-binding polypeptide comprises two or three VHH domains that bind canine PD-1; and an Fc region. In some such embodiments, each VHH domain comprises a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8. In some embodiments, each VHH domain independently comprises the amino acid sequence of SEQ ID NO: 9, 10, 11, 12, or 13.

In various embodiments, an Fc region included in a canine PD-1-binding polypeptide is a canine Fc region, or is derived from a canine Fc region.

In some embodiments, an Fc region included in a canine PD-1-binding polypeptide is derived from a canine Fc region, and comprises a three amino acid deletion in the lower hinge corresponding to IgGB E233, M234, and L235, as numbered by Kabat, herein referred to as “xEML.” In some embodiments, an Fc region included in a canine PD-1-binding polypeptide is derived from a canine Fc region, and comprises two substitutions, D265A and N297A, as numbered by Kabat, herein referred to as “DANA”. In some embodiments, an Fc region included in a canine PD-1 binding polypeptide is derived from a canine Fc region, and comprises both the xEML three amino acid deletion and the DANA substitutions, herein referred to as “xEML-DANA”. Fc xEML-DANA polypeptides do not engage FcyRs and thus are referred to as “effector silent” or “effector null;” however, in some embodiments, xEML-DANA Fc regions bind FcRn and therefore such embodiments have transcytosis associated with FcRn mediated recycling and extended half-life relative to polypeptides that do not comprise an Fc region that binds FcRn.

Nonlimiting exemplary Fc regions that may be used in a canine PD-1-binding polypeptide include Fc regions comprising the amino acid sequences of SEQ ID NOs: 19 and 20.

In some embodiments, a canine PD-1-binding polypeptide comprises one VHH domain and an Fc region, wherein the VHH domain comprises the amino acid sequence of SEQ ID NO: 9, 10, 11, 12, or 13 and the Fc region is fused to the C-terminus of the VHH domain. In some embodiments, a canine PD-1-binding polypeptide that comprises one VHH domain and an Fc region comprises or consists of the amino acid sequence of SEQ ID NO: 14, 15, 16, 17, or 18.

### Exemplary Activities of PD-1-Binding Polypeptides

In various embodiments, the canine PD-1-binding polypeptides provided herein are antagonists of canine PD-1 activity. Antagonist activity may be determined, in some embodiments, using the methods provided in the Examples herein, such as using 293, 293FS, or CHO cells expressing canine PD-1.

In some embodiments, the canine PD-1-binding polypeptides provided herein decrease and/or block binding of canine PD-L1 to canine PD-1 in vitro and/ or in vivo. In some embodiments, a canine PD-1-binding polypeptide provided herein decreases binding of canine PD-L1 to canine PD-1 in vitro. In some embodiments, the PD-1-binding polypeptide decreases binding of canine PD-L1 to canine PD-1 by at least 50%, 60%, 70%, 80%, or at least 90%. The decrease in binding of canine PD-L1 to canine PD-1 may be determined by any method in the art, such as for example, the methods provided in the Examples herein. A nonlimiting exemplary assay, described herein, comprises incubating a canine PD-1-binding polypeptide for 1 hour at room temperature in a PBS buffer with 3-4 nM canine PD-L1-hFc fusion (Sino Biological) and untransfected 293FS cells or transfected 293FS cells expressing full length canine PD-1 (SEQ ID NO: 1). A fluorescent anti-Fc specific secondary antibody is used to detect canine PD-1-binding polypeptide bound to PD-1 by flow cytometry using an Intellicyte iQue analyzer. Mean fluorescence intensity is plotted for each concentration of canine PD-1-binding polypeptide tested.

### Polypeptide Expression and Production

Nucleic acid molecules comprising polynucleotides that encode a canine PD-1-binding polypeptide are provided. Thus, in various embodiments, nucleic acid molecules are provided that encode a VHH domain that binds canine PD-1 and comprises a CDR1 comprising the amino acid sequence of SEQ ID NO: 6, a CDR2 comprising the amino acid sequence of SEQ ID NO: 7, and a CDR3 comprising the amino acid sequence of SEQ ID NO: 8. In some embodiments, nucleic acid molecules are provided that encode a canine PD-1-binding polypeptide that comprises at least one, such as one, two, three, or four VHH domains. In various embodiments, the nucleic acid molecule further encodes an Fc region, such as an Fc region of SEQ ID NO: 19 or 20. In some embodiments, a nucleic acid molecule is provided that encodes a canine PD-1-binding polypeptide that comprises at least one VHH domain and an Fc region, wherein the VHH domain comprises the amino acid sequence of SEQ ID NO: 9, 10, 11, 12, or 13 and the Fc region is fused to the C-terminus of the VHH domain. In some embodiments, a nucleic acid molecule is provided that encodes a canine PD-1-binding polypeptide comprising or consisting of the amino acid sequence of SEQ ID NO: 14, 15, 16, 17, or 18. In any of the foregoing embodiments, the nucleic acid molecule may also encode a leader sequence that directs secretion of the canine PD-1-binding polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the canine PD-1-binding polypeptides described herein are provided. Such vectors include, but are not limited to, DNA vectors, phage vectors, viral vectors, retroviral vectors, etc. In some embodiments, a vector is selected that is optimized for expression of polypeptides in a desired cell type, such as 293, 293FS, or CHO or CHO-derived cells, or in NSO cells. Exemplary such vectors are described, for example, in Running Deer et al., Biotechnol. Prog. 20:880-889 (2004).

In some embodiments, a canine PD-1-binding polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293FS and 293-6E cells; CHO cells, including CHO-S, DG44. Lec13 CHO cells, and FUT8 CHO cells; PER.C6^{®} cells (Crucell); and NSO cells. In some embodiments, the PD-1-binding polypeptides may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a canine PD-1-binding polypeptide described herein is provided. The canine PD-1-binding polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a PD-1-binding polypeptide that comprises an Fc region. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

In some embodiments, the canine PD-1-binding polypeptide is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, canine PD-1-binding polypeptides prepared by the methods described above are provided. In some embodiments, the canine PD-1-binding polypeptide is prepared in a host cell. In some embodiments, the canine PD-1-binding polypeptide is prepared in a cell-free system. In some embodiments, the canine PD-1-binding polypeptide is purified. In some embodiments, a cell culture media comprising a canine PD-1-binding polypeptide is provided.

In some embodiments, compositions comprising antibodies prepared by the methods described above are provided. In some embodiments, the composition comprises a canine PD-1-binding polypeptide prepared in a host cell. In some embodiments, the composition comprises a canine PD-1-binding polypeptide prepared in a cell-free system. In some embodiments, the composition comprises a purified canine PD-1-binding polypeptide. Exemplary methods of treating diseases using PD-1-binding polypeptides

In some embodiments, methods of treating disease in a subject comprising administering a canine PD-1-binding polypeptide are provided. Such diseases include any disease that would benefit from decreased PD-1 activity in T cells and/or increased T cell activity. In some embodiments, methods for treating cancer in a subject are provided. The method comprises administering to the subject an effective amount of a canine PD-1-binding polypeptide provided herein. Such methods of treatment may be in mammals, including canines. Nonlimiting exemplary cancers that may be treated with canine PD-1-binding polypeptides provided herein include lymphoma, hemangiosarcoma, mast cell carcinoma, melanoma, osteosarcoma, mammary cancer, renal cell carcinoma, and non-small cell lung cancer, high grade lymphoma, histiocytic sarcoma, malignant histiocytosis, urothelial carcinoma, oral squamous cell carcinoma; basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; myeloma; neuroblastoma; oral cavity cancer; ovarian cancer; pancreatic cancer; prostate cancer; retinoblastoma; rhabdomyosarcoma; rectal cancer, cancer of the respiratory system; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin’s lymphoma; non-Hodgkin’s lymphoma; B-cell lymphoma; low grade/follicular non-Hodgkin’s lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom’s macroglobulinemia; chronic lymphocytic leukemia (CLL); acute lymphoblastic leukemia (ALL); Hairy cell leukemia; and chronic myeloblastic leukemia.

The canine PD-1-binding polypeptides can be administered as needed to subjects. Determination of the frequency of administration can be made by persons skilled in the art, such as an attending veterinarian based on considerations of the condition being treated, age of the subject being treated, severity of the condition being treated, general state of health of the subject being treated and the like. In some embodiments, an effective dose of a canine PD-1-binding polypeptide is administered to a subject one or more times. In some embodiments, an effective dose of a canine PD-1-binding polypeptides is administered to the subject daily, semiweekly, weekly, every two weeks, once a month, etc. An effective dose of a canine PD-1-binding polypeptide is administered to the subject at least once. In some embodiments, the effective dose of a canine PD-1-binding polypeptides may be administered multiple times, including multiple times over the course of at least a month, at least six months, or at least a year.

In some embodiments, pharmaceutical compositions are administered in an amount effective for treating (including prophylaxis of) cancer. The therapeutically effective amount is typically dependent on the weight of the subject being treated, his or her physical or health condition, the extensiveness of the condition to be treated, or the age of the subject being treated. In general, antibodies may be administered in an amount in the range of about 0.05 mg/kg body weight to about 100 mg/kg body weight per dose. In some embodiments, antibodies may be administered in an amount in the range of about 10 µg/kg body weight to about 100 mg/kg body weight per dose. In some embodiments, antibodies may be administered in an amount in the range of about 50 µg/kg body weight to about 5 mg/kg body weight per dose. In some embodiments, antibodies may be administered in an amount in the range of about 100 µg/kg body weight to about 10 mg/kg body weight per dose. In some embodiments, antibodies may be administered in an amount in the range of about 100 µg/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 0.05 mg/kg body weight to about 20 mg/kg body weight per dose. In some embodiments, antibodies may be administered in an amount in the range of about 0.05 mg/kg body weight to about 10 mg/kg body weight per dose. In some embodiments, antibodies may be administered in an amount in the range of about 5 mg/kg body weight or lower, for example less than 4, less than 3, less than 2, or less than 1 mg/kg of the antibody.

In some embodiments, canine PD-1-binding polypeptides can be administered in vivo by various routes, including, but not limited to, intravenous, intra-arterial, parenteral, intraperitoneal or subcutaneous. The appropriate formulation and route of administration may be selected according to the intended application.

In some embodiments, a therapeutic treatment using a canine PD-1-binding polypeptide is achieved by increasing T-cell proliferation and/or activation. In some embodiments, increasing T-cell proliferation and/or activation inhibits growth of cancer.

### Pharmaceutical Compositions

In some embodiments, compositions comprising canine PD-1-binding polypeptides are provided in formulations with a wide variety of pharmaceutically acceptable carriers (see, for example, Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7^{th} ed., Lippencott Williams and Wilkins (2004); Kibbe et al., Handbook of Pharmaceutical Excipients, 3^{rd} ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, a pharmaceutical composition comprises a canine PD-1-binding polypeptide at a concentration of at least 10 mg/mL, 20 mg/mL, 30 mg/mL, 40 mg/mL, 50 mg/mL, 60 mg/mL, 70 mg/mL, 80 mg/mL, 90 mg/mL, 100 mg/mL, 125 mg/mL, 150 mg/mL, 175 mg/mL, 200 mg/mL, 225 mg/mL, or 250 mg/mL.

### Combination Therapy

Canine PD-1-binding polypeptides can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

### Nonlimiting Exemplary Methods of Diagnosis and Treatment

In some embodiments, the methods described herein are useful for evaluating a subject and/or a specimen from a subject (e.g. a subject having cancer). In some embodiments, evaluation is one or more of diagnosis, prognosis, and/or response to treatment.

In some embodiments, the methods described herein comprise evaluating a presence, absence, or level of a protein. In some embodiments, the methods described herein comprise evaluating a presence, absence, or level of expression of a nucleic acid. The compositions described herein may be used for these measurements. For example, in some embodiments, the methods described herein comprise contacting a specimen of the tumor or cells cultured from the tumor with a therapeutic agent as described herein.

In some embodiments, the evaluation may direct treatment (including treatment with the antibodies described herein). In some embodiments, the evaluation may direct the use or withholding of adjuvant therapy after resection. Adjuvant therapy, also called adjuvant care, is treatment that is given in addition to the primary, main or initial treatment. By way of non-limiting example, adjuvant therapy may be an additional treatment usually given after surgery where all detectable disease has been removed, but where there remains a statistical risk of relapse due to occult disease. In some embodiments, the antibodies are used as an adjuvant therapy in the treatment of a cancer. In some embodiments, the antibodies are used as the sole adjuvant therapy in the treatment of a cancer. In some embodiments, the antibodies described herein are withheld as an adjuvant therapy in the treatment of a cancer. For example, if a subject is unlikely to respond to an antibody described herein or will have a minimal response, treatment may not be administered in the interest of quality of life and to avoid unnecessary toxicity from ineffective chemotherapies. In such cases, palliative care may be used.

In some embodiments the molecules are administered as a neoadjuvant therapy prior to resection. In some embodiments, neoadjuvant therapy refers to therapy to shrink and/or downgrade the tumor prior to any surgery. In some embodiments, neoadjuvant therapy means chemotherapy administered to subjects having cancer prior to surgery. In some embodiments, neoadjuvant therapy means an antibody is administered to subjects having cancer prior to surgery. Types of cancers for which neoadjuvant chemotherapy is commonly considered include, for example, breast, colorectal, ovarian, cervical, bladder, and lung. In some embodiments, the antibodies are used as a neoadjuvant therapy in the treatment of a cancer. In some embodiments, the use is prior to resection.

In some embodiments, the tumor microenvironment contemplated in the methods described herein is one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T-cells; macrophages; neutrophils; and other immune cells located proximal to a tumor.

### Kits

Also provided are articles of manufacture and kits that include any of the canine PD-1-binding polypeptides as described herein, and suitable packaging. In some embodiments, the invention includes a kit with (i) a canine PD-1-binding polypeptide, and (ii) instructions for using the kit to administer the canine PD-1-binding polypeptide to a subject.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting a subject suitable or treatment.

The containers may be unit doses, bulk packages (e.g., multi-dose packages) or sub-unit doses. For example, kits may also be provided that contain sufficient dosages of molecules disclosed herein to provide effective treatment for a subject for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous suspension of antibody.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Binding of Single-Domain Antibodies to Canine PD-1

Single domain antibodies (sdAbs) comprising VHH domains that bind canine PD-1 were developed, including sdAbs comprising VHH domains having an amino acid sequences selected from SEQ ID NOs: 2-5. Five caninized versions of the VHH domain of PD1-14 were made, having the amino acid sequences of SEQ ID NOs: 9-13, respectively. The sdAbs listed in Table 2, comprising a PD1-5, PD1-8, PD1-13, or PD1-14 VHH domain, or a caninized version of the PD1-14 VHH domain and a canine IgGB Fc region comprising xEML-DANA mutations (SEQ ID NO: 20), were tested for binding to canine PD-1.

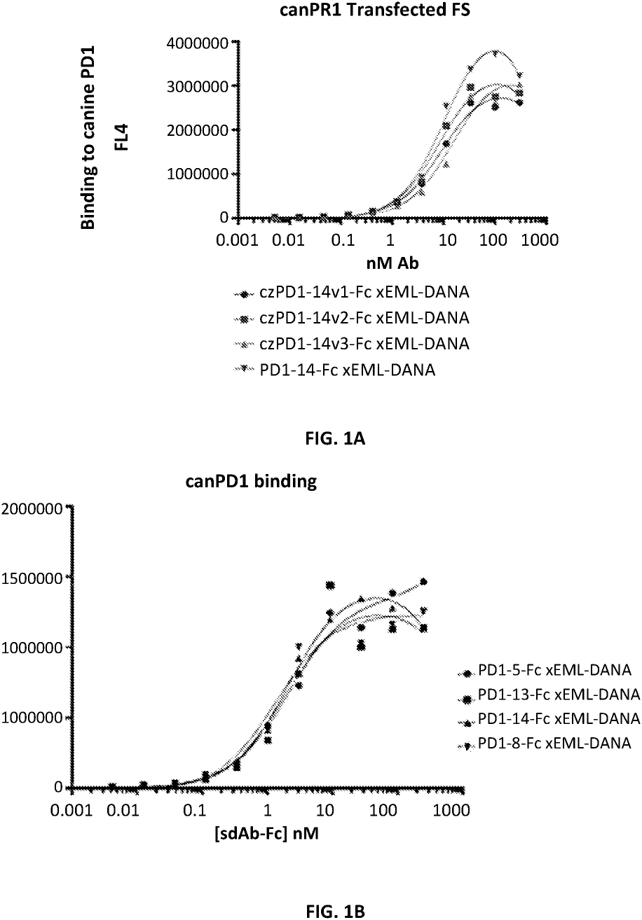

Binding of the sdAbs to canine PD-1 was determined as follows. Transiently transfected 293FS cells that express full length canine PD-1 and untransfected 293FS cells were plated at 50,000 cells/well in separate wells. Antibodies were titrated serially diluted 1:3 starting at 400 nM, and detected with anti-canine Fc 647 secondary antibody. Flow cytometric analysis was performed on an Intellicyte iQue analyzer and fluorescence was plotted as median fluorescence intensity. As shown in FIGS. 1A and 1B, the seven antibodies tested specifically bound to canine PD-1. The affinities (K_{DS}) of the sdAbs for canine PD-1 are listed in Table 2 below.

TABLE 2

| sdAb | Affinity (nM) | SEQ ID NO(s). |
|---|---|---|
| czPD1-14v1-Fc xEML-DANA | 4.151 | 14 |
| czPD1-14v2-Fc xEML-DANA | 6.647 | 15 |
| czPD1-14v3-Fc xEML-DANA | 3.243 | 16 |
| PD1-14-Fc xEML-DANA | 8.82 | 21 |
| PD1-5-Fc xEML-DANA | <10 nM | VHH: 5; Fc region: 20 |
| PD1-8-Fc xEML-DANA | <10 nM | VHH: 2; Fc region: 20 |
| PD1-13-Fc xEML-DANA | <10 nM | 31 |

### Example 2: Single-Domain Antibodies That Bind Canine PD-1 Block Binding of Canine PD-1 to PD-L1

Binding of canine PD-1 to PD-L1 in the presence of the sdAbs described in Example 1 was determined as follows. Each sdAb was incubated for 1 hour at room temperature in a PBS buffer, with 3 or 4 nM canine PD-L1-hFc fusion (Sino Biological) and untransfected 293FS cells or transfected 293FS cells expressing full length canine PD-1 (SEQ ID NO: 1). A fluorescent anti-Fc specific secondary antibody was used to detect sdAb bound to PD-1 by flow cytometry using an Intellicyte iQue analyzer. Mean fluorescence intensity was plotted for each concentration of sdAb tested.

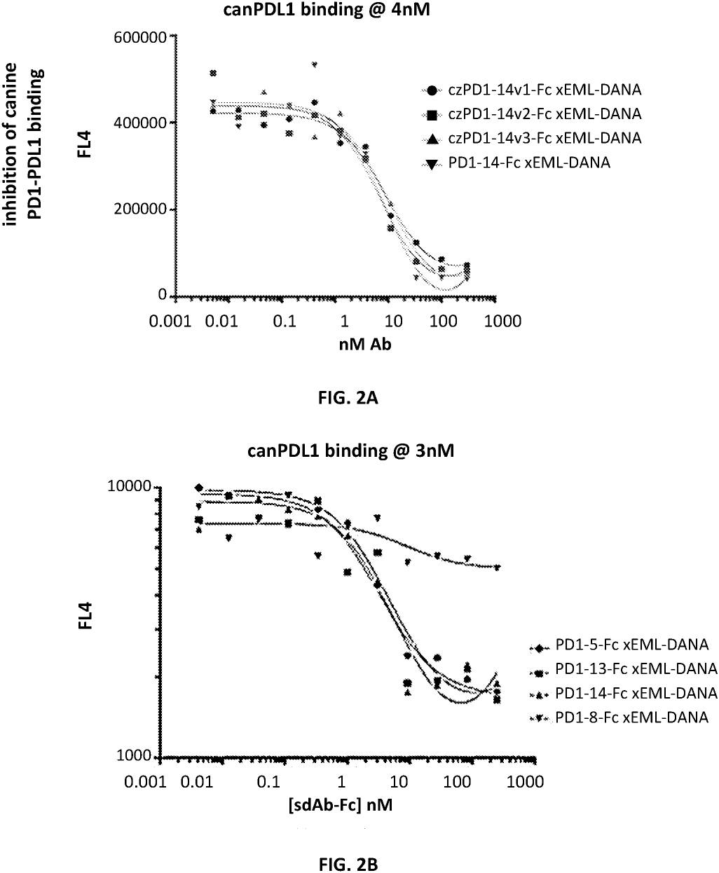

As shown in FIGS. 2A and 2B, with the exception of PD1-8, all antibodies tested blocked canine PD-1 binding to 3 or 4 nM PD-L1-hFc in a dose-dependent manner, with comparable potencies.

### Example 3: Binding of Single-Domain Antibodies to Canine Fc Receptor Components

Binding of canine Fc receptor components CD16, CD32, and CD64 to sdAbs comprising an xEML-DANA Fc region was tested. The tested sdAbs are listed in Table 3. A canine CD19 sdAb comprising a wild type canine IgGB Fc region (SEQ ID NO: 19) was used as a positive control.

The binding of the sdAbs to the canine Fc receptor components was determined as follows. Transiently transfected 293FS cells that express full length canine CD16, CD32, CD64 were plated at 50,000 cells/well in separate wells. The sdAbs were serially diluted 1:3 starting at 250 nM, and detected with anti-canine Fc 647 secondary antibody A fluorescent anti-Fc specific secondary antibody was used to detect bound sdAb by flow cytometry using an Intellicyte iQue analyzer. Mean fluorescence intensity was plotted for each concentration of antibody tested.

TABLE 3

| sdAb | SEQ ID NO. |
|---|---|
| cCD19-4A8-Fc | Fc region: SEQ ID NO: 19 |
| PD1-14-Fc xEML-DANA | 21 |
| PD1-13-Fc xEML-DANA | 31 |

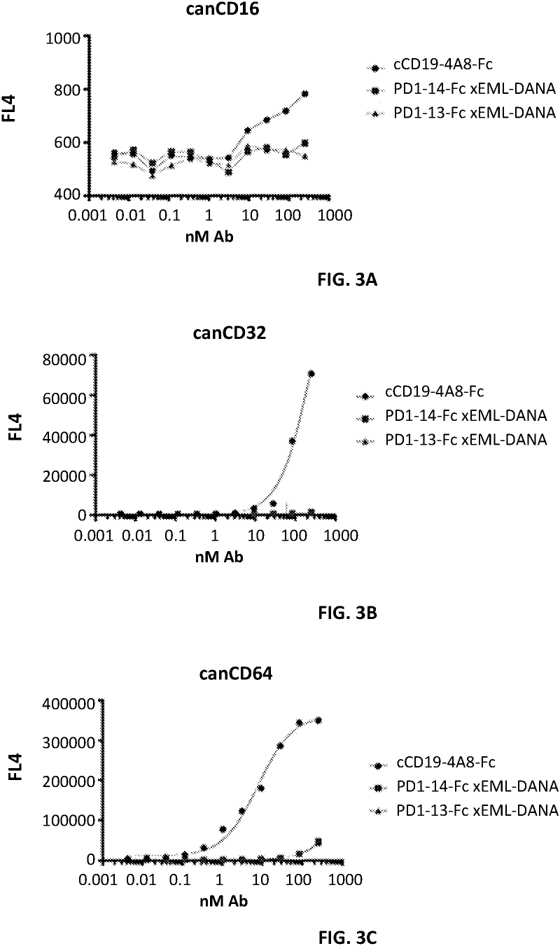

As shown in FIGS. 3A-C, the sdAbs comprising the xEML-DANA mutations in the Fc region exhibited greatly reduced binding to Fc receptor components CD16 (FIG. 3A) and CD64 (FIG. 3C) relative to the antibody comprising the wild type Fc region. Moreover, the sdAbs comprising the xEML-DANA mutations in the Fc region did not exhibit any binding to Fc receptor component CD32 (FIG. 3B).

### Example 4: T Cell Activation by sdAbs That Bind Canine PD-1

Activation of CD4+ and CD8+ T cells by sdAbs comprising a VHH domain that binds canine PD-1 and an Fc region comprising xEML and DANA mutations (SEQ ID NO: 20) was tested. The tested sdAbs are listed in Table 4. 0.25×10^6 canine PBMCs (previously frozen, thawed freshly) were plated per well in a 96-well U-bottom plate. The cells were incubated with an sdAb in FACS buffer (100 nM, 1:5) for 30 minutes at 4° C. The cells were then washed once and incubated in a surface staining mix comprising anti-dog CD3-FITC (clone CA17.2A12 1:50), anti-dog CD8-A700 (Clone:YCATE55.9 1:50) and anti-dog CD4-PE/Cy7 (Clone YKIX302.9 1:50), as well as PI (1:2000) and anti-dog A647 (1:500) for 20 minutes at room temperature. The cells were then washed a final time and analyzed on a Sony SA3800 Analyzer. Background corrected mean fluorescence intensity was plotted for each concentration of antibody tested and used to calculate the EC₅₀ values shown in Table 4 below.

TABLE 4

| sdAbs | EC₅₀ (nM) | | SEQ ID NO |
|---|---|---|---|
| | CD4 T cells | CD8 T cells | |
| PD1-14-Fc xEML-DANA | 1.703 | 0.764 | 21 |
| czPD1-14v2-Fc xEML-DANA | 9.148 | 27.25 | 15 |

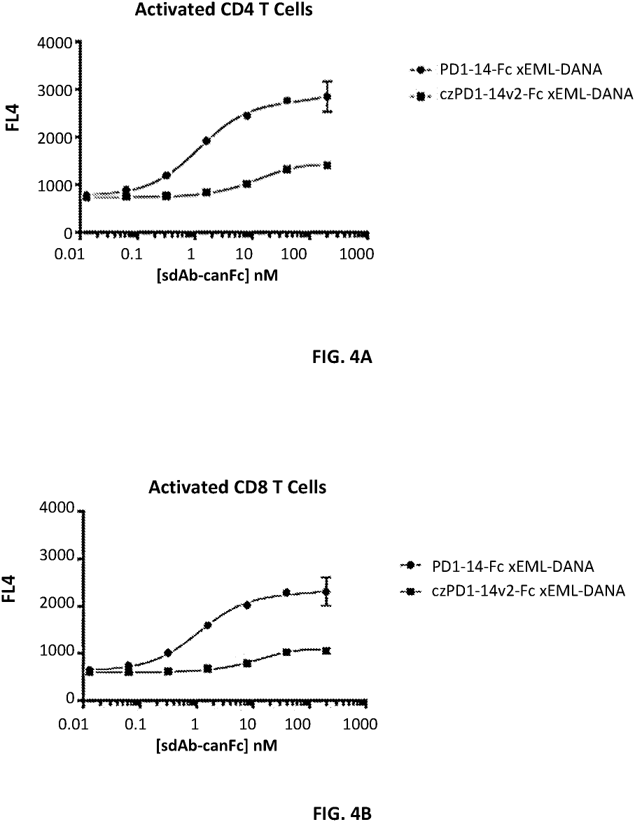

As shown in Table 4 and in FIGS. 4A-B, the tested sdAbs activated CD4 (FIG. 4A) and CD8 (FIG. 4B) T cells in a dose dependent manner.

### Example 5: Pharmacokinetics of an sdAb That Binds Canine PD-1

The pharmacokinetics of an sdAb comprising a VHH domain that binds canine PD-1 (SEQ ID NO: 3) and an Fc region comprising xEML and DANA mutations (Fc domain: SEQ ID NO: 20; sdAb: SEQ ID NO: 21) were tested in male beagles 9 months of age or greater with body weight of 9.4-11.9 kg. The dogs were fasted, then the antibody (Groups 2, 3, and 4) or vehicle control (Group 1) was administered via intravenous infusion twice to each dog, with three weeks between each infusion, at 0.5 mg/kg/dose (Group 2), 1.5 mg/kg/dose (Group 3), or 4.5 mg/kg/dose (Group 4), with a dose volume of 1 mL/kg, as shown in Table 5. Plasma samples from the cephalic vein were taken 2, 8, 24, 48, 96, 144, 312, and 480 hours post-infusion, and clinical observations were made at 1, 2, 4, 8, 12, 24, 36, and 48 hours post-dose and at least twice daily thereafter.

For PK analysis, plasma samples were stored at -40° C. until analyzed by ELISA. The ELISA was a modified version of the Acro Biosystems competitive ELISA Assay Kit for Anti-PD-1 h-mAB in Mouse Serum (Catalogue Number: EPM-V1). In the modified version, the following changes were made: 1) the kit recommended normal mouse serum was replaced with a pool of normal canine plasma, 2) the kit supplied human PD-1 was substituted with an alternate recombinant human PD-1, and 3) the kit supplied streptavidin- Horse Radish Peroxidase (HRP) reagent was replaced with a commercial streptavidin-HRP reagent. Preliminary experiments indicated that the modified kit was appropriate for measuring the canine anti-PD-1 mAb in canine plasma. Briefly, canine plasma from study subjects was mixed and incubated per assay instructions with kit supplied biotinylated anti-PD-1 antibody prior to addition to ELISA plates previously coated with recombinant human PD-1 and blocked. After appropriate incubation, plates were washed, incubated with the commercial streptavidin-HRP reagent, washed again, incubated with TMB-based substrate, stopped by addition of 1N HCl, and finally read at OD450 nm in a plate reader. Percent binding was calculated by comparing the OD values of sample wells (or standard) to wells containing only normal canine plasma (total binding activity). Known concentrations of the canine anti-PD-1 mAb diluted in normal canine plasma were used to generate the standard curves on each ELISA plate.

Pharmacokinetic evaluation based on ELISA quantification of plasma concentrations was completed using Phoenix 64 WinNonlin, Build 8.1.0.3530 following a non-compartmental approach consistent with the IV infusion route of administration.

The concentration of the sdAb in the plasma of each blood sample was measured by ELISA, and the apparent terminal elimination half-life (T_{½}), area under the curve versus time from the start of dose administration to the time of the last quantifiable concentration (AUCₗₐₛₜ), systemic clearance (C1), and volume of distribution for the sdAb at steady state (Vss) were calculated. The Evaluation showed the mean half-life of anti-PD-1 sdAB was 67.8 to 89.8 hrs. The mean half-life of anti-PD-1 sdAB for the 1st and 2nd IV infusion was 67.8 to 79.7 and 77.8 to 89.8 hrs, respectively. The clearance (mean 1.63 — 2.47 mL/hr/kg) and volume of distribution (mean 128 — 202 mL/kg) were low. The exposure (AUC) increased proportionally with dose from 0.5 to 4.5 mg/kg and there was no accumulation after two infusions.

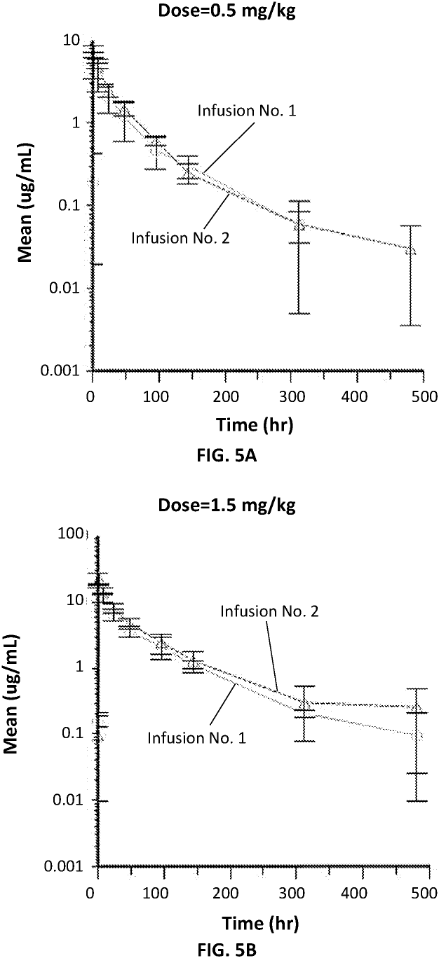

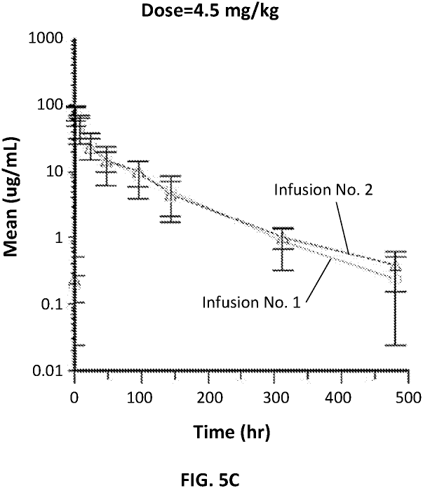

Based on the absence of test article-related observations or changes in clinical pathology, flow cytometry, or cytokine levels, anti-PD-1 sdAb, was well tolerated when administered as two intravenous (IV) infusions at all doses and administration timepoints. The results are summarized in Table 5 below and in FIGS. 5A-C.

TABLE 5

| Group (No. of dogs) | Formulation (mg/mL) | Infusion no. | Dose level (mg/kg) | Mean T_{½} (hr) | Mean AUCₗₐₛₜ (hr*µg/mL) | Mean C1 (mL/hr/kg) | Mean Vₛₛ (mL/kg) |
|---|---|---|---|---|---|---|---|
| 2 (3) | 0.5 | 1 | 0.5 | 78.8 | 205 | 2.47 | 202 |
| | | 2 | | 83.2 | 251 | 1.96 | 139 |
| 3 (3) | 1.5 | 1 | 1.5 | 79.7 | 727 | 2.03 | 172 |
| | | 2 | | 77.8 | 915 | 1.66 | 145 |
| 4 (3) | 4.5 | 1 | 4.5 | 67.8 | 3026 | 1.85 | 128 |
| | | 2 | | 89.8 | 2963 | 1.63 | 134 |

Table of Certain Sequences

| SEQ ID NO | Description | Sequence |
|---|---|---|
| 1 | Canine PD-1 | MGSRRGPWPL VWAVLQLGWW PGWLLDSPDR PWSPLTFSPA QLTVQEGENA TFTCSLADIP DSFVLNWYRL SPRNQTDKLA AFQEDRIEPG RDRRFRVTRL PNGRDFHMSI VAARLNDSGI YLCGAIYLPP NTQINESPRA ELSVTERTLE PPTQSPSPPP RLSGQLQGLV IGVTSVLVGV LLLLLLTWVL AAVFPRATRG ACVCGSEDEP LKEGPDAAPV FTLDYGELDF QWREKTPEPP APCAPEQTEY ATIVFPGRPA SPGRRASASS LQGAQPPSPE DGPGLWPL |
| 2 | PD1-8 VHH | QVQLQQSGGGLVQAGGSLRVSCAASGRTFSSYGMGWFRQAPGKEREFVAAISWSGGTQY YADSAKGRFTISRDNAKNTVYLQMNSLKPEDTAVYYCADYTDYVVYRPQEIGYWGQGTQV TVKP |
| 3 | PD1-14 VHH | QVQLQQSGGGLAQAGGSLRLSCAASGRTVSIYAMGWFRQAPGKEREFVAGIGWNGGTTY YADSVEGRFTISRDHAKNTAYLQMNSLKPEDTAVYYCAAQESAAGTLGDYWGRGTQVTVK P |
| 4 | PD1-13 VHH | QVTLKESGGGLVQAGGSLRLSCAASGRTEIIYAMGWFRQAPGKEREFVAGIGWSGGTTY YADSVEGRFTISRDSAKNTVYLQMRSLKPEDTAVYYCAAQDSVAGTLGDYWGQGTQVTVK P |
| 5 | PD1-5 VHH | QVTLRESGGGLVQPGGSLRLSCAASGLTFGLYAMTNWFRQAPGKDREGISCISSSDGSTI YADSVKGRFTASRDNAKDTMYLQMNNLNPEDTAVYYCATDYETRCDYGLRLRDRTAYWG PGTQVTVKP |
| 6 | PD1-14 CDR1 | GRTVSIYAMG |
| 7 | PD1-14 CDR2 | GIGWNGGTTY |
| 8 | PD1-14 CDR3 | QESAAGTLGDY |
| 9 | czPD1-14v1 VHH | EVQLVESGGGEVKPGGSLRLSCVASGRTVSIYAMGWFRQAPGKEREFVAGIGWSGGTTYY ADAVKGRFTISRDNAKNTVYLQMSSLRAEDTAVYYCAAQESAAGTLGDYWGQGTQVTVKP |
| 10 | czPD1-14v2 VHH | EVQLVESGGGEVKPGGSLRLSCVASGRTVSIYAMGWFRQAPGKEREFVAGIGWNGGTTYY ADAVKGRFTISRDNAKNTVYLQMSSLRAEDTAVYYCAAQESAAGTLGDYWGQGTQVTVKP |
| 11 | czPD-1-14v3 VHH | EVQLVESGGGEVKPGGSLRLSCVASGRTVSIYAMGWFRQAPGKEREFVAGIGWNGGTTYY ADAVKGRFTISRDHAKNTVYLQMSSLRAEDTAVYYCAAQESAAGTLGDYWGQGTQVTVKP |
| 12 | czPD-1-14v4 VHH | EVQLVESGGGEVKPGGSLRLSCVASGRTVSIYAMGWFRQAPGKEREFVAGIGWNGGTTYY ADAVKGRFTISRDHAKNTAYLQMSSLRAEDTAVYYCAAQESAAGTLGDYWGQGTQVTVKP |
| 13 | czPD-1-14v5 VHH | EVQLVESGGGEVKPGGSLRLSCAASGRTVSIYAMGWFRQAPGKEREFVAGIGWNGGTTYY ADAVEGRFTISRDHAKNTAYLQMNSLRAEDTAVYYCAAQESAAGTLGDYWGQGTQVTVKP |
| 14 | czPD1-14v1-Fc xEML-DANA | EVQLVESGGGEVKPGGSLRLSCVASGRTVSIYAMGWFRQAPGKEREFVAGIGWSGGTTYY ADAVKGRFTISRDNAKNTVYLQMSSLRAEDTAVYYCAAQESAAGTLGDYWGQGTQVTVKP GGGGENGRVPRPPDCPKCPAPGGPSVFIFPPKPKDTLLIARTPEVTCVVVALDPEDPEVQ ISWFVDGKQMQTAKTQPREEQFAGTYRVVSVLPIGHQDWLKGKQFTCKVNNKALPSPIER TISKARGQAHQPSVYVLPPSREELSKNTVSLTCLIKDFFPPDIDVEWQSNGQQEPESKYR TTPPQLDEDGSYFLYSKLSVDKSRWQRGDTFICAVMHEALHNHYTQKSLSHSPGK |
| 15 | czPD1-14v2-Fc | EVQLVESGGGEVKPGGSLRLSCVASGRTVSIYAMGWFRQAPGKEREFVAGIGWNGGTTYY ADAVKGRFTISRDNAKNTVYLQMSSLRAEDTAVYYCAAQESAAGTLGDYWGQGTQVTVKP GGGGENGRVPRPPDCPKCPAPGGPSVFIFPPKPKDTLLIARTPEVTCVVVALDPEDPEVQ |
| | xEML-DANA | ISWFVDGKQMQTAKTQPREEQFAGTYRVVSVLPIGHQDWLKGKQFTCKVNNKALPSPIER TISKARGQAHQPSVYVLPPSREELSKNTVSLTCLIKDFFPPDIDVEWQSNGQQEPESKYR TTPPQLDEDGSYFLYSKLSVDKSRWQRGDTFICAVMHEALHNHYTQKSLSHSPGK |
| 16 | czPD-1-14v3-Fc xEML-DANA | EVQLVESGGGEVKPGGSLRLSCVASGRTVSIYAMGWFRQAPGKEREFVAGIGWNGGTTYY ADAVKGRFTISRDHAKNTVYLQMSSLRAEDTAVYYCAAQESAAGTLGDYWGQGTQVTVKP GGGGENGRVPRPPDCPKCPAPGGPSVFIFPPKPKDTLLIARTPEVTCVVVALDPEDPEVQ ISWFVDGKQMQTAKTQPREEQFAGTYRVVSVLPIGHQDWLKGKQFTCKVNNKALPSPIER TISKARGQAHQPSVYVLPPSREELSKNTVSLTCLIKDFFPPDIDVEWQSNGQQEPESKYR TTPPQLDEDGSYFLYSKLSVDKSRWQRGDTFICAVMHEALHNHYTQKSLSHSPGK |
| 17 | czPD-1-14v4-Fc xEML-DANA | EVQLVESGGGEVKPGGSLRLSCVASGRTVSIYAMGWFRQAPGKEREFVAGIGWNGGTTYY ADAVKGRFTISRDHAKNTAYLQMSSLRAEDTAVYYCAAQESAAGTLGDYWGQGTQVTVKP GGGGENGRVPRPPDCPKCPAPGGPSVFIFPPKPKDTLLIARTPEVTCVVVALDPEDPEVQ ISWFVDGKQMQTAKTQPREEQFAGTYRVVSVLPIGHQDWLKGKQFTCKVNNKALPSPIER TISKARGQAHQPSVYVLPPSREELSKNTVSLTCLIKDFFPPDIDVEWQSNGQQEPESKYR TTPPQLDEDGSYFLYSKLSVDKSRWQRGDTFICAVMHEALHNHYTQKSLSHSPGK |
| 18 | czPD-1-14v5-Fc xEML-DANA | EVQLCVESGGGEVKPGGSLRSCAASGRTVSIYAMGWFRQAPGKEREFIVAGIGWNGGTTYY ADAVEGRFTISRDHAKNTAYLQMNSLRAEDTAVYYCAAQESAAGTLGDYWGQGTQVTVKP GGGGENGRVPRPPDCPKCPAPGGPSVFIFPPKPKDTLLIARTPEVTCVVVALDPEDPEVQ ISWFVDGKQMQTAKTQPREEQFAGTYRVVSVLPIGHQDWLKGKQFTCKVNNKALPSPIER TISKARGQAHQPSVYVLPPSREELSKNTVSLTCLIKDFFPPDIDVEWQSNGQQEPESKYR TTPPQLDEDGSYFLYSKLSVDKSRWQRGDTFICAVMHEALHNHYTQKSLSHSPGK |
| 19 | Canine IgGB Fc region | ENGRVPRPPDCPPCPAPEMLGGPSVFIFPPKPKDTLLIARTPEVTCVVVDLDPEDPEVQI SWFVDGKQMQTAKTQPREEQFNGTYRVVSVLPIGHQDWLKGKQFTCKVNNKALPSPIERT ISKARGQAHQPSVYVLPPSREELSKNTVSLTCLIKDFFPPDIDVEWQSNGQQEPESKYRT TPPQLDEDGSYFLYSKLSVDKSRWQRGDTFICAVMHEALHNHYTQKSLSHSPGK |
| 20 | Canine IgGB Fc region (xEML-DANA) | ENGRVPRPPDCPKCPAPGGPSVFIFPPKPKDTLLIARTPEVTCVVVALDPEDPEVQISWF VDGKQMQTAKTQPREEQFAGTYRVVSVLPIGHQDWLKGKQFTCKVNNKALPSPIERTISK ARGQAHQPSVYVLPPSREELSKNTVSLTCLIKDFFPPDIDVEWQSNGQQEPESKYRTTPP QLDEDGSYFLYSKLSVDKSRWQRGDTFICAVMHEALHNHYTQKSLSHSPGK |
| 21 | PD1-14-Fc xEML-DANA | QVQLQQSGGGLAQAGGSLRLSCAASGRTVSIYAMGWFRQAPGKEREFVAGIGWNGGTTYY ADSVEGRFTISRDHAKNTAYLQMNSLKPEDTAVYYCAAQESAAGTLGDYWGRGTQVTVKP GGGGENGRVPRPPDCPKCPAPGGPSVFIFPPKPKDTLLIARTPEVTCVVVALDPEDPEVQ ISWFVDGKQMQTAKTQPREEQFAGTYRVVSVLPIGHQDWLKGKQFTCKVNNKALPSPIER TISKARGQAHQPSVYVLPPSREELSKNTVSLTCLIKDFFPPDIDVEWQSNGQQEPESKYR TTPPQLDEDGSYFLYSKLSVDKSRWQRGDTFICAVMHEALHNHYTQKSLSHSPGK |
| 22 | PD1-8 CDR1 | GRTFSSYGMG |
| 23 | PD1-8 CDR2 | AISWSGGTQY |
| 24 | PD1-8 CDR3 | YTDYVVYRPQEIGY |
| 25 | PD1-13 CDR1 | GRTEIIYAMG |
| 26 | PD1-13 CDR2 | GIGWSGGTTY |
| 27 | PD1-13 CDR3 | QDSVAGTLGDY |
| 28 | PD1-5 CDR1 | GLTFGLYAMT |
| 29 | PD1-5 CDR2 | GIGWSGGTTY |
| 30 | PD1-5 CDR3 | TDYETRCDYGLRLRDRTAY |
| 31 | PD1-13-Fc xEML-DANA | QVTLKESGGGLVQAGGSLRLSCAASGRTEIIYAMGWFRQAPGKEREFVAGIGWSGGTTY YADSVEGRFTISRDSAKNTVYLQMRSLKPEDTAVYYCAAQDSVAGTLGDYWGQGTQVTVK PGGGGENGRVPRPPDCPKCPAPGGPSVFIFPPKPKDTLLIARTPEVTCVVVALDPEDPEV QISWFVDGKQMQTAKTQPREEQFAGTYRVVSVLPIGHQDWLKGKQFTCKVNNKALPSPIE RTISKARGQAHQPSVYVLPPSREELSKNTVSLTCLIKDFFPPDIDVEWQSNGQQEPESKY RTTPPQLDEDGSYFLYSKLSVDKSRWQRGDTFICAVMHEALHNHYTQKSLSHSPGK |

What is claimed is:

1. A polypeptide comprising at least one caninized VHH domain that binds canine PD-1, wherein the polypeptide comprises a canine Fc region, and wherein at least one VHH domain that binds canine PD-1 comprises:
a) a CDR1 comprising the amino acid sequence of SEQ ID NO: 6, a CDR2 comprising the amino acid sequence of SEQ ID NO: 7, a CDR3 comprising the amino acid sequence of SEQ ID NO: 8;
b) a CDR1 comprising the amino acid sequence of SEQ ID NO: 22, a CDR2 comprising the amino acid sequence of SEQ ID NO: 23, a CDR3 comprising the amino acid sequence of SEQ ID NO: 24;
c) a CDR1 comprising the amino acid sequence of SEQ ID NO: 25, a CDR2 comprising the amino acid sequence of SEQ ID NO: 26, a CDR3 comprising the amino acid sequence of SEQ ID NO: 27; or
d) a CDR1 comprising the amino acid sequence of SEQ ID NO: 28, a CDR2 comprising the amino acid sequence of SEQ ID NO: 29, a CDR3 comprising the amino acid sequence of SEQ ID NO: 30.

2. A polypeptide comprising at least one VHH domain that binds canine PD-1, wherein the polypeptide comprises a canine Fc region, and wherein at least one VHH domain that binds canine PD-1 comprises an amino acid sequence selected from SEQ ID NOs: 2-5.

3. The polypeptide of claim 1 or 2, comprising one VHH domain.

4. The polypeptide of claim 1 or 2, comprising two VHH domains, wherein the two VHH domains are the same or different.

5. The polypeptide of claim 1 or 2, comprising three VHH domains, wherein the three VHH domains are the same or different.

6. The polypeptide of any one of the preceding claims, wherein each VHH domain binds canine PD-1.

7. The polypeptide of any one of claims 1 to 6, wherein the canine Fc region is a canine IgG Fc region.

8. The polypeptide of claim 7, wherein the canine Fc region is a canine IgGB Fc region.

9. The polypeptide of claim 7 or claim 8, wherein amino acids E233, M234, and L235 of the Fc region, as determined by Kabat numbering, are deleted.

10. The polypeptide of any one of claims 7 to 9, wherein the Fc region comprises D265A and N297A substitutions, as determined by Kabat numbering.

11. The polypeptide of any one of claims 1 to 8, wherein the Fc region comprises the amino acid sequence of SEQ ID NO: 19.

12. The polypeptide of any one of claims 1 to 10, wherein the canine Fc region comprises the amino acid sequence of SEQ ID NO: 20.

13. The polypeptide of any one of claims 1 to 12, wherein at least one VHH domain that binds canine PD-1 comprises CDR1 comprising the amino acid sequence of SEQ ID NO: 6, a CDR2 comprising the amino acid sequence of SEQ ID NO: 7, a CDR3 comprising the amino acid sequence of SEQ ID NO: 8.

14. The polypeptide of any one of claims 1 to 13, wherein at least one VHH domain that binds canine PD-1 comprises the amino acid sequence of SEQ ID NO: 3.

15. The polypeptide of any one of claims 1 to 10 or 12 to 14, wherein the polypeptide comprises the amino acid sequence of SEQ ID NO: 21.

16. The polypeptide of any one of claims 1 or 3 to 13, wherein at least one VHH domain that binds canine PD-1 is caninized.

17. The polypeptide of claim 16, wherein at least one caninized VHH domain that binds canine PD-1 comprises a CDR1 comprising the amino acid sequence of SEQ ID NO: 6, a CDR2 comprising the amino acid sequence of SEQ ID NO: 7, a CDR3 comprising the amino acid sequence of SEQ ID NO: 8, and an amino acid sequence having at least 90%, 95%, 96%, 97%, 98%, 99%, or 100% identity an amino acid sequence selected from SEQ ID NOs: 9-13.

18. The polypeptide of claim 16 or claim 17, wherein at least one caninized VHH domain that binds canine PD-1 comprises an amino acid sequence selected from SEQ ID NOs: 9-13.

19. The polypeptide of any one of claim 16 to 18, wherein the polypeptide comprises an amino acid sequence selected from SEQ ID NOs: 14-18.

20. The polypeptide of any one of claims 1 to 19, which forms a dimer under physiological conditions.

21. The polypeptide of any one of claims 1 to 20, wherein the polypeptide decreases canine PD-1 binding to canine PD-L1 in vitro and/or in vivo.

22. The polypeptide of claim 21, wherein the polypeptide decreases canine PD-1 binding to canine PD-L1 in vitro by at least 50%, 60%, 70%, 80%, or at least 90%.

23. The polypeptide of any one of claims 1 to 22, which is an antagonist of canine PD-1 biological activity.

24. The polypeptide of any one of claims 1 to 23, wherein the polypeptide binds canine PD-1 with an affinity (K_{D}) of less than 100 nM, less than 50 nM, less than 25 nM, or less than 10 nM.

25. A pharmaceutical composition comprising the polypeptide of any one of claims 1 to 24 and a pharmaceutically acceptable carrier.

26. An isolated nucleic acid that encodes the polypeptide of any one of claims 1 to 24.

27. A vector comprising the nucleic acid of claim 26.

28. A host cell comprising the nucleic acid of claim 26 or the vector of claim 27.

29. A host cell that expresses the polypeptide of any one of claims 1 to 24.

30. A method of producing the polypeptide of any one of claims 1 to 24 comprising incubating the host cell of claim 28 or claim 29 under conditions suitable for expression of the polypeptide.

31. The method of claim 30, further comprising isolating the polypeptide.

32. A method of activating canine CD4+ T cells and/or canine CD8+ T cells comprising contacting the T cells with the polypeptide of any one of claims 1 to 24.

33. The method of claim 32, wherein the T cells are in vitro.

34. The method of claim 32, wherein the T cells are in vivo.

35. A method of treating cancer in a canine comprising administering to a canine with cancer a pharmaceutically effective amount of the polypeptide of any one of claims 1 to 24 or the pharmaceutical composition of claim 25.

36. The method of claim 35, wherein the cancer is selected from lymphoma, hemangiosarcoma, mast cell carcinoma, melanoma, osteosarcoma, mammary cancer, renal cell carcinoma, and a non-small cell lung cancer.

37. The method of claim 35 or 36, further comprising an additional anti-cancer therapy.

38. The method of claim 37, wherein the additional anti-cancer therapy comprises at least one therapy selected from cancer resection, radiation therapy, and administration of an additional anti-cancer agent.


SEQUENCE LISTING (31 sequences)

SEQ ID NO: 1 - protein, 288 aa, Canis familiaris
MGSRRGPWPLVWAVLQLGWWPGWLLDSPDRPWSPLTFSPAQLTVQEGENATFTCSLADIPDSFVLNWYRLSPRNQTDKLAAFQEDRIEPGRDRRFRVTRLPNGRDFHMSIVAARLNDSGIYLCGAIYLPPNTQINESPRAELSVTERTLEPPTQSPSPPPRLSGQLQGLVIGVTSVLVGVLLLLLLTWVLAAVFPRATRGACVCGSEDEPLKEGPDAAPVFTLDYGELDFQWREKTPEPPAPCAPEQTEYATIVFPGRPASPGRRASASSLQGAQPPSPEDGPGLWPL

SEQ ID NO: 2 - protein, 123 aa, Artificial Sequence
  PD1-8 VHH
QVQLQQSGGGLVQAGGSLRVSCAASGRTFSSYGMGWFRQAPGKEREFVAAISWSGGTQYYADSAKGRFTISRDNAKNTVYLQMNSLKPEDTAVYYCADYTDYVVYRPQEIGYWGQGTQVTVKP

SEQ ID NO: 3 - protein, 120 aa, Artificial Sequence
  PD1-14 VHH
QVQLQQSGGGLAQAGGSLRLSCAASGRTVSIYAMGWFRQAPGKEREFVAGIGWNGGTTYYADSVEGRFTISRDHAKNTAYLQMNSLKPEDTAVYYCAAQESAAGTLGDYWGRGTQVTVKP

SEQ ID NO: 4 - protein, 120 aa, Artificial Sequence
  PD1-13 VHH
QVTLKESGGGLVQAGGSLRLSCAASGRTEIIYAMGWFRQAPGKEREFVAGIGWSGGTTYYADSVEGRFTISRDSAKNTVYLQMRSLKPEDTAVYYCAAQDSVAGTLGDYWGQGTQVTVKP

SEQ ID NO: 5 - protein, 127 aa, Artificial Sequence
  PD1-5 VHH
QVTLRESGGGLVQPGGSLRLSCAASGLTFGLYAMTWFRQAPGKDREGISCISSSDGSTIYADSVKGRFTASRDNAKDTMYLQMNNLNPEDTAVYYCATDYETRCDYGLRLRDRTAYWGPGTQVTVKP

SEQ ID NO: 6 - protein, 10 aa, Artificial Sequence
  PD1-14 CDR1
GRTVSIYAMG

SEQ ID NO: 7 - protein, 10 aa, Artificial Sequence
  PD1-14 CDR2
GIGWNGGTTY

SEQ ID NO: 8 - protein, 11 aa, Artificial Sequence
  PD1-14 CDR3
QESAAGTLGDY

SEQ ID NO: 9 - protein, 120 aa, Artificial Sequence
  czPD1-14v1 VHH
EVQLVESGGGEVKPGGSLRLSCVASGRTVSIYAMGWFRQAPGKEREFVAGIGWSGGTTYYADAVKGRFTISRDNAKNTVYLQMSSLRAEDTAVYYCAAQESAAGTLGDYWGQGTQVTVKP

SEQ ID NO: 10 - protein, 120 aa, Artificial Sequence
  czPD1-14v2 VHH
EVQLVESGGGEVKPGGSLRLSCVASGRTVSIYAMGWFRQAPGKEREFVAGIGWNGGTTYYADAVKGRFTISRDNAKNTVYLQMSSLRAEDTAVYYCAAQESAAGTLGDYWGQGTQVTVKP

SEQ ID NO: 11 - protein, 120 aa, Artificial Sequence
  czPD-1-14v3 VHH
EVQLVESGGGEVKPGGSLRLSCVASGRTVSIYAMGWFRQAPGKEREFVAGIGWNGGTTYYADAVKGRFTISRDHAKNTVYLQMSSLRAEDTAVYYCAAQESAAGTLGDYWGQGTQVTVKP

SEQ ID NO: 12 - protein, 120 aa, Artificial Sequence
  czPD-1-14v4 VHH
EVQLVESGGGEVKPGGSLRLSCVASGRTVSIYAMGWFRQAPGKEREFVAGIGWNGGTTYYADAVKGRFTISRDHAKNTAYLQMSSLRAEDTAVYYCAAQESAAGTLGDYWGQGTQVTVKP

SEQ ID NO: 13 - protein, 120 aa, Artificial Sequence
  czPD-1-14v5 VHH
EVQLVESGGGEVKPGGSLRLSCAASGRTVSIYAMGWFRQAPGKEREFVAGIGWNGGTTYYADAVEGRFTISRDHAKNTAYLQMNSLRAEDTAVYYCAAQESAAGTLGDYWGQGTQVTVKP

SEQ ID NO: 14 - protein, 355 aa, Artificial Sequence
  czPD1-14v1-Fc xEML-DANA
EVQLVESGGGEVKPGGSLRLSCVASGRTVSIYAMGWFRQAPGKEREFVAGIGWSGGTTYYADAVKGRFTISRDNAKNTVYLQMSSLRAEDTAVYYCAAQESAAGTLGDYWGQGTQVTVKPGGGGENGRVPRPPDCPKCPAPGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 15 - protein, 355 aa, Artificial Sequence
  czPD1-14v2-Fc xEML-DANA
EVQLVESGGGEVKPGGSLRLSCVASGRTVSIYAMGWFRQAPGKEREFVAGIGWNGGTTYYADAVKGRFTISRDNAKNTVYLQMSSLRAEDTAVYYCAAQESAAGTLGDYWGQGTQVTVKPGGGGENGRVPRPPDCPKCPAPGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 16 - protein, 355 aa, Artificial Sequence
  czPD-1-14v3-Fc xEML-DANA
EVQLVESGGGEVKPGGSLRLSCVASGRTVSIYAMGWFRQAPGKEREFVAGIGWNGGTTYYADAVKGRFTISRDHAKNTVYLQMSSLRAEDTAVYYCAAQESAAGTLGDYWGQGTQVTVKPGGGGENGRVPRPPDCPKCPAPGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 17 - protein, 355 aa, Artificial Sequence
  czPD-1-14v4-Fc xEML-DANA
EVQLVESGGGEVKPGGSLRLSCVASGRTVSIYAMGWFRQAPGKEREFVAGIGWNGGTTYYADAVKGRFTISRDHAKNTAYLQMSSLRAEDTAVYYCAAQESAAGTLGDYWGQGTQVTVKPGGGGENGRVPRPPDCPKCPAPGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 18 - protein, 355 aa, Artificial Sequence
  czPD-1-14v5-Fc xEML-DANA
EVQLVESGGGEVKPGGSLRLSCAASGRTVSIYAMGWFRQAPGKEREFVAGIGWNGGTTYYADAVEGRFTISRDHAKNTAYLQMNSLRAEDTAVYYCAAQESAAGTLGDYWGQGTQVTVKPGGGGENGRVPRPPDCPKCPAPGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 19 - protein, 234 aa, Artificial Sequence
  Canine IgGB Fc region
ENGRVPRPPDCPPCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 20 - protein, 231 aa, Artificial Sequence
  Canine IgGB Fc region (xEML-DANA)
ENGRVPRPPDCPKCPAPGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 21 - protein, 355 aa, Artificial Sequence
  PD1-14-Fc xEML-DANA
QVQLQQSGGGLAQAGGSLRLSCAASGRTVSIYAMGWFRQAPGKEREFVAGIGWNGGTTYYADSVEGRFTISRDHAKNTAYLQMNSLKPEDTAVYYCAAQESAAGTLGDYWGRGTQVTVKPGGGGENGRVPRPPDCPKCPAPGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 22 - protein, 10 aa, Artificial Sequence
  PD1-8 CDR1
GRTFSSYGMG

SEQ ID NO: 23 - protein, 10 aa, Artificial Sequence
  PD1-8 CDR2
AISWSGGTQY

SEQ ID NO: 24 - protein, 14 aa, Artificial Sequence
  PD1-8 CDR3
YTDYVVYRPQEIGY

SEQ ID NO: 25 - protein, 10 aa, Artificial Sequence
  PD1-13 CDR1
GRTEIIYAMG

SEQ ID NO: 26 - protein, 10 aa, Artificial Sequence
  PD1-13 CDR2
GIGWSGGTTY

SEQ ID NO: 27 - protein, 11 aa, Artificial Sequence
  PD1-13 CDR3
QDSVAGTLGDY

SEQ ID NO: 28 - protein, 10 aa, Artificial Sequence
  PD1-5 CDR1
GLTFGLYAMT

SEQ ID NO: 29 - protein, 10 aa, Artificial Sequence
  PD1-5 CDR2
GIGWSGGTTY

SEQ ID NO: 30 - protein, 19 aa, Artificial Sequence
  PD1-5 CDR3
TDYETRCDYGLRLRDRTAY

SEQ ID NO: 31 - protein, 355 aa, Artificial Sequence
  PD1-13-Fc xEML-DANA
QVTLKESGGGLVQAGGSLRLSCAASGRTEIIYAMGWFRQAPGKEREFVAGIGWSGGTTYYADSVEGRFTISRDSAKNTVYLQMRSLKPEDTAVYYCAAQDSVAGTLGDYWGQGTQVTVKPGGGGENGRVPRPPDCPKCPAPGGPSVFIFPPKPKDTLLIARTPEVTCVVVALDPEDPEVQISWFVDGKQMQTAKTQPREEQFAGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK